(19) United States
(12) Patent Application Publication
(10) Pub. No.: US 2007/0027180 A1
(43) Pub. Date: Feb. 1, 2007

(54) PROCESS FOR PREPARING ZOLPIDEM

(76) Inventors: Pratap Reddy Padi, Hyderabad 500 076, A.P. (IN); Satyanarayana Bollikonda, Hyderabad 500 050, A.P. (IN); Ananda Mohan Jasty, Hyderabad 500 072, A.P. (IN); Ranga Reddy Tamma, Hyderabad 500 072, A.P. (IN); Saravanan Mohanarangam, Villupuram 605 108, Tamil Nadu (IN); Sumalatha Yasareni, Khammam 507 115, A.P. (IN); Gowri Shanker Rupakala, Hyderabad 500 007, A.P. (IN); Ghosh Debasish, Secunderabad 500 017, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/532,959

(22) Filed: Sep. 19, 2006

Related U.S. Application Data
(60) Provisional application No. 60/788,821, filed on Apr. 3, 2006.

(30) Foreign Application Priority Data
Sep. 19, 2005 (IN) 1318/CHE/2005

(51) Int. Cl. A61K 31/4745; C07D 471/02

(52) U.S. Cl. 514/303; 546/117

(57) ABSTRACT

A process for preparing zolpidem.

### INTRODUCTION TO THE INVENTION

The present invention relates to a process for the preparation of zolpidem and intermediates thereof.

Zolpidem has the chemical name N,N,6-trimethyl-2-p-tolyl-imidazo[1,2,-a]pyridine-3-acetamide (hereinafter referred to by the adopted name “zolpidem”), and has the structural formula shown as Formula I.

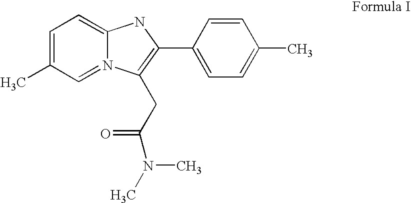

Zolpidem is a non-benzodiazepine hypnotic of the imidazopyridine class. It is used in the treatment of insomnia. It has a rapid onset of action and has a short elimination half-life.

Zolpidem is commercially available in the form of a salt with L-(+)-tartaric acid wherein the molar ratio of zolpidem to tartaric acid in the salt is 2:1 (conventionally called “zolpidem tartrate”) under the trade name AMBIEN™ as immediate release tablets in 5 mg and 10 mg strengths for oral administration. It is also available as AMBIEN CR™ 6.25 mg, being recommended for elderly patients, and 12.5 mg recommended for adults.

European Patent No. 50563 discloses zolpidem and related compounds. The patent also gives a general process for their preparation involving the reduction of a nitrile intermediate to its corresponding amide followed by hydrolysis of the amide to get an acid. The acid in turn is converted to the final compound by condensing with an amine.

The aforementioned process involves numerous synthetic steps, and isolation of intermediates at all of the stages. A scheme with a fewer number of steps, which eliminates the isolation of unstable and hazardous intermediates, would be useful.

European Patent 1104765 describes a process for the preparation of an intermediate for zolpidem involving the reaction of toluene with α-halo-acetyl halide under Friedel-Crafts acylation conditions followed by condensation of the halogenated intermediate with 2-amino-5-methyl pyridine.

The process involves the usage of alpha-halo acetyl halide which is difficult to handle and unstable, which makes the process too sensitive to be scaled up.

Processes for the preparation of zolpidem and its tartarate salt have also been described in U.S. Application Publication Nos. US 2005/0054669, US 2004/0010146, US 2002/0183522, and US 2002/0019528, International Application Publication Nos. WO 2004/087703, WO 2004/058758, WO 2002/014306, WO 2001/080857, and WO 2001/038327, European Patent No. 1292304, and U.S. Pat. No. 6,958,417.

The present invention provides a process for the preparation of zolpidem and its salts with a reduced number of synthetic steps, eliminating the need to isolate certain intermediates, which are difficult to handle. The process of the present invention can be practiced on an industrial scale, and also can be carried out without sacrifice of overall yield.

### SUMMARY OF THE INVENTION

The present invention relates to a process for the preparation of zolpidem and intermediates thereof.

One aspect of the present invention provides a process for the preparation of 6-methyl-2-(4-methyl phenyl) imidazo [1,2-a] pyridine-3-acetic acid of Formula IX comprising the steps of:

a) reacting 3-(N,N-dimethylaminomethyl)-6-methyl-2-(4-methyl-phenyl) imidazo [1,2-α]pyridine of Formula VI with methyl halide to get a quaternary halide salt;

b) reacting the quaternary halide salt of 3-(N,N-dimethylaminomethyl)-6-methyl-2-(4-methyl-phenyl) imidazo [1,2-α]pyridine of Formula VII with an alkali cyanide to yield 6-methyl-2-(4-methyl phenyl) imidazo [1,2-α] pyridine-3-acetonitrile of Formula VIII; and

c) hydrolysis of 6-methyl-2-(4-methyl phenyl) imidazo [1,2-α] pyridine-3-acetonitrile of Formula VIII in the presence of a suitable base to provide 6-methyl-2-(4-methyl phenyl) imidazo [1,2-α] pyridine-3-acetic acid of Formula IX.

Suitably, one or more sequential steps are carried out without isolating intermediate compounds. In an embodiment of the invention, step b) is carried out without isolating the intermediate, followed by isolation of the compound of Formula IX.

Another aspect of the present invention provides a process for the preparation of zolpidem compound of Formula I comprising reacting 6-methyl-2-(4-methyl phenyl) imidazo [1,2-α] pyridine-3-acetic acid of Formula IX with dimethyl amine in the presence of a suitable reagent.

Yet another aspect of the present invention provides a process for the synthesis of 3-(N,N-dimethylaminomethyl)-6-methyl-2-(4-methyl-phenyl)-imidazo[1,2-α]pyridine of Formula VI comprising the steps of:

a) halogenation of 4-methyl acetophenone of Formula II to yield 4-methyl haloacetophenone of Formula III; and

b) condensation of 4-methyl haloacetophenone of Formula III with 2-amino 5-methyl pyridine of Formula IV to yield 2-(4-methyl phenyl) 6-methyl-[1,2-a] imidazopyridine of Formula V.

In an embodiment, step a) is carried out in-situ to avoid isolation of the halogenated intermediate of Formula III.

An embodiment of the invention comprises a process for preparing zolpidem or a salt thereof, comprising reacting 3-(N,N-dimethylaminomethyl)-6-methyl-2-(4-methylphenyl)-imidazo-[1,2-a]-pyridine with an alkyl halide and, without isolating any intermediate, reacting with an alkali metal cyanide, then reacting with a base to form 6-methyl-2-(4-methylphenyl)-imidazo-[1,2-a]-pyridine-3-acetic acid.

Another embodiment of the invention comprises zolpidem or a salt thereof, containing about 0.0001 to about 0.5%, or about 0.0001 to about 0.1 percent, by high performance liquid chromatography of any of the impurities having the formulae:

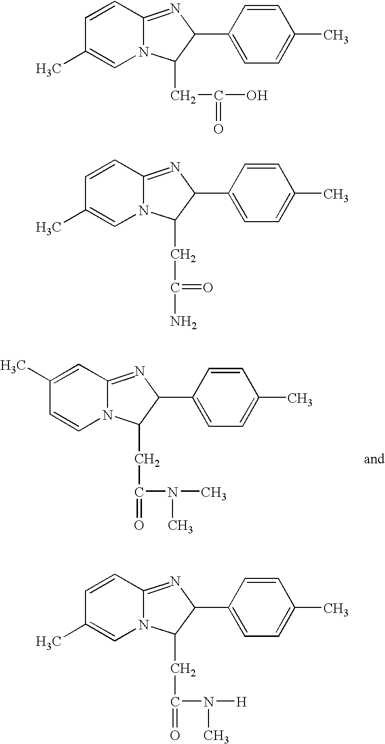

### BRIEF DESCRIPTION OF THE DRAWINGS

FIG. 1 is a schematic representation of a process for preparing zolpidem.

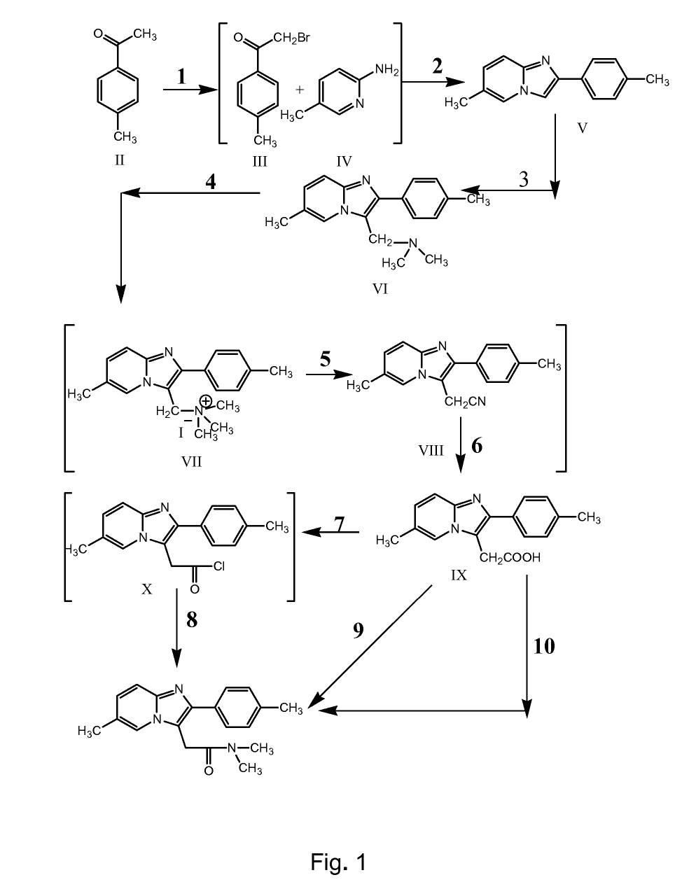

FIG. 2 is a high performance liquid chromatography chromatogram showing zolpidem and impurity peaks.

### DETAILED DESCRIPTION OF THE INVENTION

The present invention relates to a process for the preparation of zolpidem and intermediates thereof.

One aspect of the present invention provides a process for the synthesis of 6-methyl-2-(4-methyl phenyl) imidazo [1,2-a] pyridine-3-acetic acid of Formula IX.

In an embodiment of the invention, a process for the preparation of 6-methyl-2-(4-methyl phenyl) imidazo [1,2-a] pyridine-3-acetic acid of Formula IX comprises:

a) quaternizing 3-(N,N-dimethylaminomethyl)-6-methyl-2-(4-methyl-phenyl) imidazo [1,2-α]pyridine of Formula VI with an alkyl halide, such as methyl iodide, to get an alkyl halide salt, such as the methyl iodide salt of Formula VII;

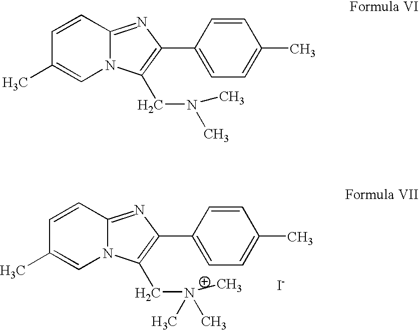

b) reacting the alkyl halide salt of 3-(N,N-dimethylaminomethyl)-6-methyl-2-(4-methyl-phenyl) imidazo [1,2-α]pyridine of Formula VII with an alkali metal cyanide to yield 6-methyl-2-(4-methyl phenyl) imidazo [1,2-a] pyridine-3-acetonitrile of Formula VIII; and

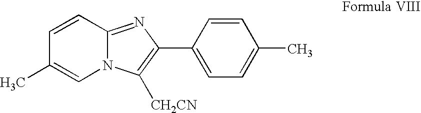

c) hydrolyzing 6-methyl-2-(4-methyl phenyl) imidazo [1,2-a] pyridine-3-acetonitrile of Formula VIII in the presence of a suitable base to provide 6-methyl-2-(4-methyl phenyl) imidazo [1,2-a] pyridine-3-acetic acid of Formula IX.

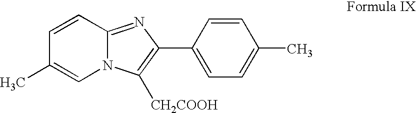

Step a) involves reacting 3-(N,N-dimethylaminomethyl)-6-methyl-2-(4-methyl-phenyl) imidazo [1,2-α]pyridine of Formula VI with an alkyl halide to get a quaternary halide salt.

Useful alkyl halides include, but are not limited to, methyl iodide, methyl chloride, methyl bromide and the like.

Suitable solvents for use in this step include, but are not limited to: alcohol solvents such as C₁-C₄ alcohols; C₂-C₆ ketone solvents such as acetone, ethyl methyl ketone, and diethyl ketone; chlorinated solvents, such as C₁-C₆ straight chain or branched chlorohydrocarbons such as dichloromethane, ethylene dichloride, chloroform, carbon tetrachloride, chloro benzene, dichlorobenzene and the like; and mixtures thereof.

Suitable temperatures for conducting the reaction range from about 10° C. to about 50° C., or from about 20° C. to about 40° C.

The alkyl halide is typically cooled to lower temperatures of below 25° C. before adding to a solution of 3-(N,N-dimethylaminomethyl)-6-methyl-2-(4-methyl-phenyl) imidazo [1,2-α]pyridine of Formula VI.

The quaternized product of Formula VII that is isolated from the reaction mass may be filtered and washed with a solvent if desired to wash out the mother liquor and optionally further dried, or the entire reaction mass can be carried forward for the next step.

Step b) involves reacting the quaternary halide salt of 3-(N,N-dimethylaminomethyl)-6-methyl-2-(4-methyl-phenyl) imidazo [1,2-α]pyridine of Formula VII with an alkali metal cyanide to yield 6-methyl-2-(4-methyl phenyl) imidazo [1,2-a] pyridine-3-acetonitrile of Formula VIII.

Suitable solvents that can be used for conducting the reaction include, but are not limited to: water; alcohol solvents like methanol, ethanol, isopropyl alcohol and the like; ketone solvents such as acetone, ethyl methyl ketone, methyl isobutyl ketone and the like; hydrocarbon solvents such as toluene, xylene and the like; nitrile solvents such as acetonitrile, propionitrile and the like; and mixtures thereof in various proportions.

Suitable temperatures for conducting the reaction range from about 30 to about 100° C., or from about 60 to about 90° C.

Suitable alkali metal cyanides, which can be used include, but are not limited to sodium cyanide, potassium cyanide and the like.

Suitably, the reaction mass containing the product 6-methyl-2-(4-methyl phenyl) imidazo [1,2-a] pyridine-3-acetonitrile of Formula VIII is used for the next stage without isolation of the product. This in situ process eliminates the handling of the cyano intermediate of Formula VIII, which is hazardous to handle.

Step c) involves hydrolysis of 6-methyl-2-(4-methyl phenyl) imidazo [1,2-a] pyridine-3-acetonitrile of Formula VIII in the presence of a suitable base to provide 6-methyl-2-(4-methyl phenyl) imidazo [1,2-a] pyridine-3-acetic acid of Formula IX;

Suitable bases that can be used for hydrolysis include, but are not limited to: hydroxides of alkali metals such as lithium hydroxide, sodium hydroxide, potassium hydroxide and the like; carbonates of alkali metals such as sodium carbonate, potassium carbonate and the like; and bicarbonates of alkali metals such as sodium bicarbonate, potassium bicarbonate and the like.

Suitable solvents which can be used include, but are not limited to: alcohol solvents such as C₁-C₄ alcohols; chlorinated solvents such as C₁-C₆ straight chain or branched chlorohydrocarbons such as dichloromethane, ethylene dichloride, chloroform, carbon tetrachloride, chloro benzene, dichlorobenzene; water; and the like, and mixtures thereof.

Suitable temperatures for conducting the reaction range from about 30 to about 100° C., or from about 70 to about 90° C.

After reaction completion, the reaction mass is taken into water and the pH is made neutral or acidic to isolate the acid of Formula IX.

Suitable acids that can be used for adjusting the pH include but are not limited to: organic acids like acetic acid, trifluoroacetic acid, para-toluene sulfonic acid, oxalic acid, formic acid and the like; and inorganic acids like sulfuric acid, hydrochloric acid, nitric acid, hydrobromic acid and like.

The separated acid compound can be filtered and washed with a suitable solvent to wash out the mother liquor.

The acid can optionally be further dried to remove the residual solvent and moisture. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods until the desired result is obtained, times from about 1 to 20 hours frequently being suitable.

One or more sequential steps can be carried out without isolating intermediate compounds. In one embodiment of the invention, step b) is carried out without isolating the intermediate, followed by isolation of the compound of Formula IX.

Another aspect of the present invention provides a process for the preparation of zolpidem of Formula I comprising reacting 6-methyl-2-(4-methyl phenyl) imidazo [1,2-a] pyridine-3-acetic acid of Formula IX with dimethylamine in the presence of a suitable reagent, in a suitable solvent.

6-methyl-2-(4-methyl phenyl) imidazo [1,2-a] pyridine-3-acetic acid of Formula IX is suitably converted to a more reactive intermediate before condensing with dimethylamine. Reagents which can be used for the purpose of providing an unstable intermediate which can easily condense with dimethylamine to form the intermediate include chlorinating agents and other complex forming reagents.

According to one embodiment the compound of Formula IX is reacted with a suitable chlorinating agent and the acid chloride thus obtained is reacted with dimethylamine in a suitable solvent to get the compound of Formula I.

According to another embodiment the acetic acid derivative of the Formula IX is reacted with a carbonyldiimidazole to form the corresponding imidazole intermediate followed by reacting with dimethylamine in a suitable solvent to obtain the desired compound of Formula I.

According to a further embodiment, the acetic acid derivative of the Formula IX is reacted with 1,3-dicyclohexylcarbodiimide in the presence of 1-hydroxybenzotriazole hydrate to form the corresponding reactive intermediate in a suitable solvent followed by condensation with dimethylamine to obtain a compound of Formula I.

Suitable chlorinating agents that can be used include, but are not limited to, oxalyl chloride, phosphorus pentachloride, phosphorus trichloride, and the like. These chlorinating agents have an advantage over thionyl chloride in that they do not form a tarry product after reaction completion that is difficult to work up.

When the reaction is carried out in the presence of 1,3-dicyclohexylcarbodiimide and 1-hydroxybenzotriazole hydrate, it provides the advantage that the reaction is less moisture sensitive, and the reagents are easy to handle.

Suitable solvents which can be used for the reaction include but are not limited to: ketones such as acetone, ethyl methyl ketone, methyl isobutyl ketone and the like; chlorinated solvents, such as C₁-C₆ straight chain or branched chlorohydrocarbons such as dichloromethane, ethylene dichloride, chloroform, carbon tetrachloride, chloro benzene, dichlorobenzene; C₁-C₆ straight chain, branched or cyclic ethers; and the like, and mixtures thereof.

Selection of solvent is important as the solvent used for the reaction determines the ease of the workup procedure. Hence, low boiling solvents like halogenated solvents and ketonic solvents are used for conducting the reaction.

The above solvents can be easily evaporated when compared to tetrahydrofuran and dimethylformamide, and the quantities of solvents used in the present process are also small, thereby reducing the cost. The solvents used can be recovered after completion of the work-up.

Suitable temperatures for conducting the reaction range from about 20° C. to about 120° C.

The product isolated may optionally be purified by slurrying or recrystallization or a combination thereof in a suitable solvent.

Recrystallization involves providing a solution of crude zolpidem in a suitable solvent and then crystallizing the solid from the solution.

Suitable solvents in which zolpidem can be dissolved for purification include but are not limited to: C₁-C₅ ketones such as acetone, ethyl methyl ketone, butanone and the like; alcohols such as ethanol, methanol, and isopropanol; ethers such as ethers such as tetrahydrofuran, 1,4-dioxane, ethyl acetate and the like; water; and mixtures thereof.

The concentration of the zolpidem in the solvent can range from 40 to 80% or more. The solution can be prepared at an elevated temperature if desired to achieve a desired concentration. Any temperature is acceptable for the dissolution as long as a clear solution of the zolpidem is obtained and is not detrimental to the drug substance chemically or physically. The solution may be brought down to room temperature for further processing if required or an elevated temperature may be used. A higher temperature will allow the precipitation of solutions with higher concentrations of zolpidem resulting in better economies of manufacture.

Zolpidem obtained above can be converted to its pharmaceutically acceptable salts by processes known in the art.

Yet another aspect of the invention provides a process for the synthesis of the intermediate 3-(N,N-dimethylaminomethyl)-6-methyl-2-(4-methyl-phenyl)-imidazo[1,2-α]pyridine of Formula VI comprising the steps of:

a) halogenation of 4-methyl acetophenone of Formula II under Friedel-Crafts conditions to yield 4-methyl haloacetophenone of Formula III; and

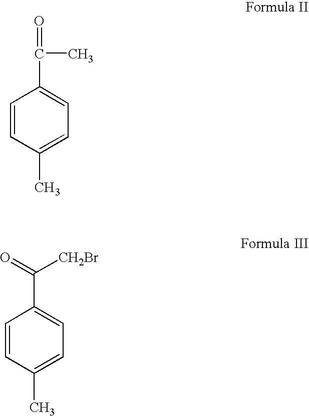

b) condensation of 4-methyl haloacetophenone of Formula III with 2-amino 5-methyl pyridine of Formula IV to yield 2-(4-methyl phenyl) 6-methyl-[1,2-a] imidazopyridine of Formula V.

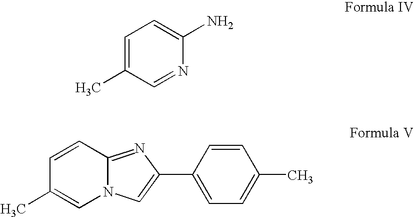

Step a) involves halogenation of 4-methyl acetophenone of Formula II under Friedel-Crafts conditions to yield 4-methyl haloacetophenone of Formula III.

Suitable halogenating agents which can be used include, but are not limited to, bromine, chlorine, and the like.

Suitable Friedel-Crafts acylating agents include, but are not limited to, aluminum chloride, ferric chloride and the like.

Suitable solvents for conducting the reaction include, but are not limited to: acetonitrile and the like; ethers such as tetrahydrofuran, 1,4-dioxane and the like; halogenated solvents such as dichloromethane, ethylene dichloride and the like; alcohols such as methanol, ethanol, isopropanol and the like; ketones such as acetone, methyl isobutyl ketone and the like; hydrocarbons such as toluene and the like; and mixtures thereof.

Suitable temperatures for conducting the reaction range from about −10° C. to about 50° C., or from about 10 to about 20° C.

The reaction mass containing 4-methyl haloacetophenone of Formula III can be directly proceeded to stage b) without isolation. This in situ process avoids the need to isolate the halogenated intermediate.

Step b) involves condensation of 4-methyl haloacetophenone of Formula III with 2-amino 5-methyl pyridine of Formula IV in the presence of a base and a suitable solvent to yield 2-(4-methyl phenyl) 6-methyl-[1,2-α] imidazopyridine of Formula V.

Suitable solvents which can be used for this step include, but are not limited to: alcohol solvents such as C₁-C₄ alcohols, chlorinated solvents, such as C₁-C₆ straight chain or branched chlorohydrocarbons such as dichloromethane, ethylene dichloride, chloroform, carbon tetrachloride, chloro benzene, dichlorobenzene and the like; and mixtures thereof.

Suitable bases which can be used for condensation include but are not limited to: hydroxides of alkali metals such as lithium hydroxide, sodium hydroxide, potassium hydroxide and the like; carbonates of alkali metals such as sodium carbonate, potassium carbonate and the like; and bicarbonates of alkali metals such as sodium bicarbonate, potassium bicarbonate and the like.

Suitable temperatures for conducting the reaction range from about 20 to about 120° C., or from about 50 to about 80° C.

The product can be isolated form the reaction mass by filtration techniques such as decantation, filtration by gravity or suction, centrifugation, and the like.

Suitably step a) is carried out in situ to avoid isolation of the halogenated intermediate of Formula III.

6-methyl-2-(4-methyl-phenyl)-imidazo[1,2-a]pyridine of Formula V obtained can be converted to 3-(N,N-dimethylaminomethyl)-6-methyl-2-(4-methyl-phenyl)-imidazo[1,2-a]pyridine of Formula VI by a process comprising reacting with dimethyl amine in the presence of formaldehyde under acidic conditions. This is exemplified for related compounds in GB 991,589, page 3, Example VII and described in lines 29-34 on page 2.

Suitable acids which can be used for providing the acidic conditions include, but are not limited to organic acids like acetic acid, formic acid, and inorganic acids like hydrochloric acid, sulfuric acid and the like.

Zolpidem obtained by the process of the present invention is substantially free of its process-related impurities. Zolpidem prepared in accordance with the present invention contains about 0.0001% to less than about 0.5%, or less than about 0.1%, of the corresponding impurities including zolpidic acid impurity, zolpidem amide impurity, zolpidem acetamide impurity, and zolpidem methyl amine impurity, as characterized by a high performance liquid chromatography (“HPLC”) chromatogram obtained from a mixture comprising the desired compound and one or more of the said impurities. The percentages for purity and impurities herein are expressed on a weight basis.

As used herein, “zolpidic acid impurity” refers to 6-methyl-2-(4-methyl phenyl) imidazo [1,2-a] pyridine-3-acetic acid, represented by Formula IX; 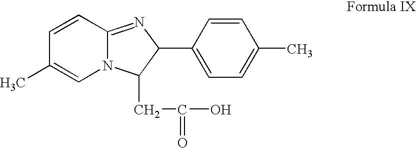
“zolpidem amide impurity” refers to 2-(4-methyl phenyl)-6-methyl imidazo [1,2-a] pyridine-3-acetamide, represented by Formula Ia; 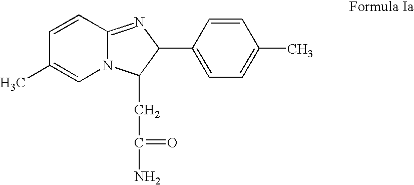
“zolpidem acetamide impurity” refers to N,N-dimethyl-2-[7-methyl-2-(4-methyl phenyl) imidazo[1,2-a]pyridine-3-yl]acetamide, represented by Formula Ib; and 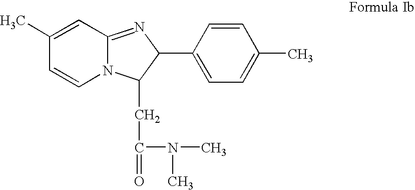
“zolpidem methyl amine impurity” refers to N,6-dimethyl-2-(4-methyl phenyl) imidazo [1,2-a] pyridine-3-acetamide, represented by Formula Ic.

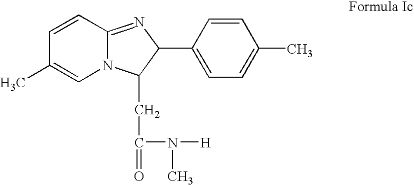

The simple and improved process of the present invention is eco-friendly, industrially well-suited, commercially viable, reproducible and cost effective with improved yield.

Certain specific aspects and embodiments of the present invention will be explained in more detail with reference to the following examples, which are provided by way of illustration only and should not be construed as limiting the scope of the invention in any manner.

### EXAMPLE 1

### Preparation of 2-(4-Methyl Phenyl) 6-Methyl-[1,2-α] Imidazo Pyridine (Formula V)

27 liters of methanol was taken into a reactor and cooled to 2.5° C. 9 kg of 4-methyl acetophenone and 0.44 kg of aluminum chloride were added to the solvent at 2° C. 11.5 kg of pre-cooled (to less than 15° C.) liquid bromine was added to the above reactor at a feed rate of 2-2.5 kg/hour. The reaction mixture was stirred at 2° C. for 30 minutes. Reaction completion was confirmed using thin layer chromatography. After completion of reaction, 9 liters of demineralized water was added to the reaction mixture at 10° C. and the reaction mixture was stirred for 50 minutes. A solution of 9 kg of sodium carbonate monohydrate in 18 liters of water was added to the reaction mixture at 17 to 18° C. The temperature of the reaction mixture was raised to 28° C. and a solution of 7.62 kg of 2-amino-5-methyl pyridine in 18 liters of water was added to the reaction mixture at 35° C. The reaction mixture was maintained at 30° C. for 3 hours. Reaction completion was confirmed using thin layer chromatography. After completion of the reaction 72 liters of water was added. The formed suspension was stirred at 29° C. for 60 minutes. The separated solid was filtered and washed with 36 liters of water to yield 23.4 kg of wet title compound.

Purity: 99% by HPLC.

### EXAMPLE 2

### Preparation of 2-(4-Methyl Phenyl)-3-Dimethyl Amino Methyl-6-Methyl-[1 2-α] Imidazo Pyridine (Formula VI)

23.45 liters of glacial acetic acid was taken into a reactor and 13.4 kg of 6-methyl-2-(4-methyl-phenyl)-imidazo [1,2-α] pyridine was added to it. The mixture was stirred at 25° C. for 60 minutes. A solution of 9.64 liters of 30% aqueous dimethylamine was added to the reactor at 25° C. 6.28 kg of 40% aqueous formaldehyde was added to the reactor at 25° C. The reaction mixture was maintained at 25° C. for 4 hours. Reaction completion was checked using thin layer chromatography. After completion of reaction, the reaction mass was cooled to 4° C. A solution of 23.47 liters of 48% aqueous sodium hydroxide solution in 54 liters of water was prepared and cooled to 25° C. The aqueous sodium hydroxide solution was added to the reaction mixture at 9° C. to adjust the pH of the reaction mass to 10.13. The temperature of the reaction mixture was raised to 29° C. and maintained for 2 hours. The separated solid was filtered and washed with 13.4 liters of water. The wet solid was taken into another reactor and 67 liters of water was added to it. The solid was slurried in the water at 33° C. for 3 hours. The solid was filtered and washed with 13.4 liters of water, then was dried at 56° C. under a vacuum of 650 mm Hg for 7 hours to yield the crude title compound. 80 liters of acetone was taken into a fresh reactor and the crude material was added to it. The mixture was heated to 55° C. and maintained for 2 hours to get a clear solution. The solution was then cooled to 2° C. and maintained for 5 hours at 2 to 3° C. The separated solid was filtered and washed with 8 liters of chilled acetone. The wet solid was dried under a vacuum of 650 mm Hg at a temperature of 60° C. to yield 11.2 kg of the title compound. (Yield: 66.5%)

Purity by HPLC: 98.9%.

### EXAMPLE 3

### Preparation of 6-Methyl-2-(4-Methyl Phenyl) Imidazo [1,2-α] Pyridine-3-ACETIC ACID (Formula IX)

80 liters of acetone and 8 kg of 2-(4-methyl phenyl)-3-dimethyl amino methyl-6-methyl-[1,2-α] imidazo pyridine were taken into a reactor and the mixture was heated to 39° C. The reaction mixture was maintained at 38 to 39° C. for 20 minutes. The reaction mass was then cooled to 26° C. Methyl iodide was pre-cooled to 15° C. and added to the reaction mass under stirring. The reaction mixture was maintained at 27 to 28° C. for 10 hours. Reaction completion was checked using thin layer chromatography. After the reaction was complete, the reaction mass was filtered and the solid was washed with 8 liters of chilled acetone. Into another reactor 60 liters of water and 1.4 kg of sodium cyanide were added. The wet solid obtained above was also added to the reactor and the reaction mass was heated to 84° C. The reaction mixture was maintained at 82 to 84° C. for 12 hours. Reaction completion was checked using high performance liquid chromatography. After the reaction was completed, the reaction mixture was cooled to 55° C. A solution of 12 liters of 48% aqueous sodium hydroxide solution in 40 liters of water was added to the reaction mass. The reaction mixture was heated to 85° C., and maintained for 3 hours. The temperature was slowly raised to 98° C. and maintained under reflux for 13 hours, 30 minutes. Reaction completion was checked using high performance liquid chromatography. After the reaction was complete, the reaction mixture was cooled to 33° C. and filtered through a Celite bed and the bed was washed with 8 liters of water. The filtrate was taken into a fresh reactor and washed with 40 liters of toluene in two equal lots. The pH of the filtrate was adjusted to 5.3 with 14 liters of acetic acid. After completion of the pH adjustment, the filtrate was maintained at 25° C. for 4 hours. The separated solid was filtered and washed with 16 liters of water in two equal lots. The wet solid was dried at 85° C. and a vacuum of 650 mm Hg for 12 hours to yield 5.6 kg of the title compound. (Yield 69.73%)

Purity by HPLC: 98.2%.

### EXAMPLE 4

### Preparation of N,N,6-Trimethyl-2-(4-Methyl Phenyl) Imidazo [1,2-α] Pyridine-3-Acetamide (Formula I)

40 liters of methylene chloride and 4 kg of phosphorus pentachloride were taken into a reactor and stirred for 10 minutes. 5 kg of 6-methyl-2-(4-methyl phenyl) imidazo [1,2-α] pyridine-3-acetic acid was added to the reactor slowly at 28° C. The reaction mixture was heated to 38° C. and maintained at that temperature for 4 hours. Reaction completion was checked using thin layer chromatography. After the reaction was completed, the reaction mass was cooled to 10° C. Dimethylamine gas was purged into the reaction mass until the reaction mass became a clear solution, then the purging was continued for 2 hours. Reaction completion was checked using thin layer chromatography. After the reaction was complete, 30 liters of the methylene chloride were distilled from the reaction solution atmospherically. 30 liters of water was added to it and heated to 85° C., and the methylene chloride was distilled completely. The reaction suspension was then cooled to 30° C. and maintained for 1 hour. pH of the suspension was adjusted to 9.6 using 3 liters of 48 % aqueous sodium hydroxide solution. The suspension was maintained at 30° C. for 2 hours. The separated solid was filtered and washed with 5 liters of water. The filtered compound was taken into another reactor and 25 liters of water were added. The mixture was slurried for one hour and then filtered. The wet compound was dried at 80° C. and a vacuum of 650 mm Hg for 5 hours to get a crude title compound.

The crude compound was charged into another reactor and 121.5 liters of acetone was added to it. The mixture was heated to 55° C. and maintained for 1 hour to get clear dissolution. 0.45 kg of activated carbon was added and maintained at the same temperature for 30 minutes. The solution was filtered and the carbon bed was washed with 4.5 liters of acetone. The filtrate was cooled to 2° C. and maintained for 2 hours. The separated solid was filtered and washed with 4.5 liters of chilled acetone. The wet compound was dried at 77° C. and a vacuum of 650 mm Hg for 5 hours to yield 3.4 kg of the title compound. (Yield 62.04%)

Purity by HPLC: 100%.

Zolpidic acid impurity content: 0.0003%.

Zolpidem amide impurity content: 0.0006%

Zolpidem methyl amine impurity content: 0.0008%

Zolpidem acetamide impurity content: 0.0016%

### EXAMPLE 5

### Alternate Route for the Preparation of N,N,6-Trimethyl-2-(4-Methyl Phenyl) Imidazo [1,2-α] Pyridine-3-Acetamide (Formula I)

58 liters of dichloromethane was taken into a reactor and 5 kg of carbonyl diimidazole was added to it. The contents were stirred for 10 minutes at 25° C. 4.8 kg of 6-methyl-2-(4-methyl phenyl) imidazo [1,2-α] pyridine-3-acetic acid was added slowly at 25° C. The reaction mixture was heated to 39° C. and maintained for 4 hours. Reaction completion was checked using thin layer chromatography. After the reaction was complete, the reaction mass was cooled to 28° C. Dimethylamine gas was purged into the reaction mass until the reaction mass became a clear solution, and the purging was continued for 2 hours. Reaction completion was checked using thin layer chromatography. After the reaction was complete, 35 liters of the methylene chloride was distilled from the reaction mass atmospherically. 29 liters of water was added and heated to 85° C., and the methylene chloride was distilled out azeotropically. The reaction mass was then cooled to 30° C. and maintained for 1 hour. pH of the reaction mass was adjusted to 9.7 using 3 liters of 48% aqueous NaOH. The suspension was maintained at 30° C. for 2 hours. The separated solid was filtered and washed with 5 liters of water. The filtered compound was taken into another reactor and 24 liters of water were added. The mixture was slurried for one hour and then filtered. The wet compound was dried at 80° C. and a vacuum of 650 mm Hg for 4 hours to get a crude title compound. The crude compound was charged into another reactor and 100 liters of acetone was added. The mixture was heated to 55° C. and maintained for 1 hour to get a clear solution. 0.4 kg of activated carbon was added and maintained at the same temperature for 30 minutes. The solution was filtered and the carbon bed was washed with 4.5 liters of acetone. The filtrate was cooled to 2° C. and maintained for 2 hours. The separated solid was filtered and washed with 4.5 liters of chilled acetone. The wet compound was dried at 70° C. and a vacuum of 690 mm Hg for 5 hours to yield 3.0 kg of the title compound. (Yield: 57.03%).

Purity by HPLC: 100%.

### EXAMPLE 6

### Alternate Route for the Preparation of N,N,6-Trimethyl-2-(4-Methyl Phenyl) Imidazo [1,2-α] Pyridine-3-Acetamide (Formula I)

5.0 g of 6-methyl-2-(4-methyl phenyl) imidazo [1,2-α] pyridine-3-acetic acid, 4.0 g of 1,3-dicyclohexylcarbodiimide, 2.5 g of 1 -hydroxybenzotriazole hydrate, and 100 ml dichloromethane were taken into a round bottom flask and stirred. Dimethylamine gas was passed through the reaction mass at 25 to 35° C. pH of the reaction mass was verified to be between 9 to 10. The reaction mixture was stirred at 25 to 35° C. until the reaction was completed. Reaction completion was confirmed by high performance liquid chromatography. After reaction completion, the reaction mass was filtered and the filter bed was washed with 25 ml dichloromethane. The dichloromethane layer was washed with a solution of 3.1 g of sodium bicarbonate in 25 ml water followed by 15 ml of water. The dichloromethane layer was distilled under vacuum completely. 50 ml water was added to the residue obtained and pH of the suspension was adjusted to about 10 using 48% aqueous sodium hydroxide solution. The compound was filtered and washed with 10 ml of water. The compound was dried in an oven at 70 to 80° C. for 5 to 6 hours to yield a crude form of the title compound.

The crude compound obtained and 100 ml acetone was added into a round bottom flask and heated to reflux. The mass was checked for clear dissolution and then 0.4 g carbon was added to the reaction solution. The solution was maintained under reflux for 15 to 30 minutes. The solution was filtered in the hot condition, and the filter bed was washed with 4 ml of acetone. The combined filtrates were taken into a round bottom flask and cooled to 0 to 5° C. The solution was maintained at 0 to 5° C. for 30 to 60 minutes. The mass was filtered and the solid was washed with 4 ml acetone. The compound was dried in an oven at 70-80° C. for 8 to 10 hours to yield 2.6 g of the title compound.

### EXAMPLE 7

### Preparation of N,N,6-Trimethyl-2-(4-Methyl Phenyl) Imidazo [1,2-α] Pyridine-3-Acetamide Hemitartrate

18 liters of methanol and 3 kg of zolpidem were taken into a reactor and stirred at 28° C. for 10 minutes. The mixture was checked for clear dissolution. After clear dissolution was obtained, the solution was filtered and the filtrate was taken into another reactor. A clear pre-filtered solution of 0.73 kg of L-(+)-tartaric acid in 12 liters of methanol was added to the filtrate at 29° C. The reaction mass was stirred at 29° C. for 60 minutes. The reaction mass was then cooled to 2° C., and maintained for 60 minutes. The reaction mass was filtered and the solid was washed with 3 liters of chilled (2° C.) methanol. The wet compound was dried at 62° C. under a vacuum of 620 mm Hg for 8 hours. The dry material was further dried in a fluidized bed drier with a drier air flow between 50 to 60%, inlet air temperature of 62° C., outlet temperature of 47° C. and a relative humidity of 20% at the outlet for 10 minutes. The dry material was micronized under an air jet pressure of 2.5 to 3.5 kg/cm², and feed air pressure of 3.5 to 4.5 kg/cm², and a relative humidity of the inlet air of 17% to yield 2.68 kg of the title compound (Yield 71.84%).

Purity by HPLC: 99.98%.

Zolpidic acid impurity content: 0.02%

Zolpidem amide content: 0.0006%

Zolpidem methyl amine impurity: 0.0008%

Zolpidem acetamide impurity: 0.0016%

### EXAMPLE 8

### Determination of Impurities in Zolpidem Tartrate

TABLE 1
HPLC conditions for detecting the levels of impurities in zolpidem tartrate.

| | |
|---|---|
| Column: | Waters Novapak C18 150 × 3.9 mm, 4μ particle size. |
| Flow rate: | 1.5 mL/minute |
| Column oven: | Ambient temperature |
| Wavelength: | 254 nm |
| Injection | 20 μl |
| volume: | |
| Run time: | 40 minutes |
| Elution: | Isocratic |
| Diluent: | Mobile phase |
| Mobile phase: | Buffer:Acetonitrile:Methanol in a ratio of 590:180:230 |
| | (v/v) |
| Buffer: | 5.6 g of orthophosphoric acid dissolved in 1000 mL of |
| | water and the pH adjusted to 5.5 with triethylamine. |

By dividing the column retention times observed for the individual impurity peaks by the retention time for the compound of interest, relative retention times (“RRT”) for the peaks are generated. The following table gives the RRT values for compounds described herein:

| COMPOUND | RRT |
|---|---|
| Zolpidic acid impurity | 0.20 |
| Zolpidem amide impurity | 0.41 |
| Zolpidem acetamide impurity | 0.85 |
| Zolpidem methyl amine | 0.56 |
| Zolpidem tartrate | 1.0 |

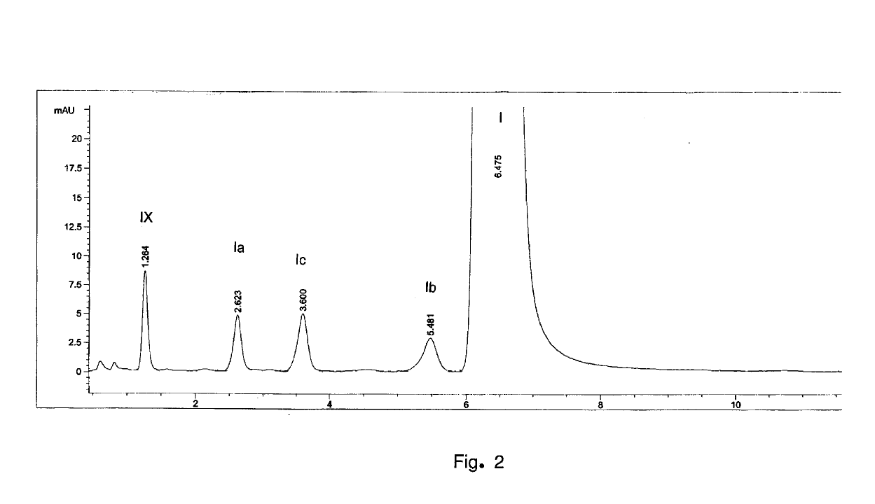

FIG. 2 is a high performance liquid chromatography chromatogram showing peaks for all of the impurity compounds, using the above-described conditions and a sample that contains zolpidem and each of the four impurities. The peaks have the indicated retention times and are labeled as follows:

“IX” is the zolpidic acid impurity.

“Ia” is the zolpidem amide impurity.

“Ib is the zolpidem acetamide impurity.

“Ic” is the zolpidem methyl amine impurity.

“I” is zolpidem.

The horizontal axis shows elapsed minutes after sample injection and the vertical axis is detector light absorption (in absorbance units÷1000).

1. A process for preparing zolpidem or a salt thereof, comprising reacting 3-(N,N-dimethylaminomethyl)-6-methyl-2-(4-methylphenyl)-imidazo-[1,2-a]-pyridine with an alkyl halide and, without isolating any intermediate, reacting with an alkali metal cyanide, then reacting with a base to form 6-methyl-2-(4-methylphenyl)-imidazo-[1,2-a]-pyridine-3-acetic acid.

2. The process of claim 1, wherein an alkyl halide comprises methyl iodide.

3. The process of claim 1, wherein 3-(N,N-dimethylaminomethyl)-6-methyl-2-(4-methylphenyl)-imidazo[1,2-a]pyridine is prepared by a process comprising condensing a 4-methyl haloacetophenone with 2-amino 5-methyl pyridine to yield 2-(4-methyl phenyl) 6-methyl-[1,2-a] imidazopyridine.

4. The process of claim 3, wherein a 4-methyl haloacetophenone is prepared by halogenating 4-methyl acetophenone in the presence of a Friedel-Crafts catalyst, and the 4-methyl haloacetophenone is not isolated before further reaction.

5. The process of claim 4, wherein halogenating is performed with bromine.

6. The process of claim 1, wherein zolpidem is prepared by a process comprising reacting 6-methyl-2-(4-methylphenyl)-imidazo-[1,2-a]-pyridine with dimethylamine and formaldehyde in an acidic medium to form 3-(N,N-dimethylaminomethyl)-6-methyl-2-(4-methylphenyl)-imidazo[1,2-a]pyridine.

7. The process of claim 1, wherein zolpidem is prepared by a process further comprising comprising amidating 6-methyl-2-(4-methylphenyl)-imidazo-[1,2-a]-pyridine-3-acetic acid.

8. The process of claim 7, wherein amidating comprises reacting with a carbonyldiimidazole to form the corresponding imidazole intermediate, followed by reacting with dimethylamine.

9. The process of claim 7, wherein amidating comprises reacting with 1,3-dicyclohexylcarbodiimide in the presence of 1-hydroxybenzotriazole hydrate, followed by condensation with dimethylamine.

10. The process of claim 7, wherein amidating is conducted in a dichloromethane solvent.

11. The process of claim 1, wherein zolpiden is reacted with an acid to form a salt.

12. Zolpidem or a salt thereof, containing about 0.0001 to about 0.5% by high performance liquid chromatography of any of the impurities having the formulae:

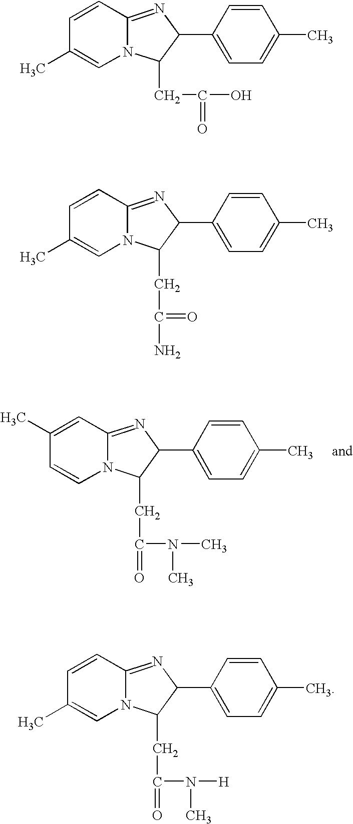

13. Zolpidem or a salt thereof of claim 12, containing about 0.0001 to about 0.1% by high performance liquid chromatography of any of the impurities having the formulae:

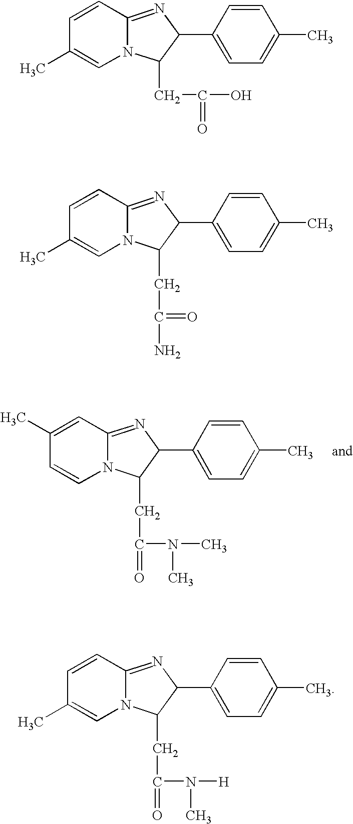

14. Zolpidem or a salt thereof of claim 12, containing less than about 0.1 percent by high performance liquid chromatography of an impurity having the formula:

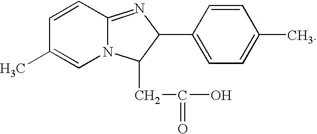

15. Zolpidem or a salt thereof of claim 12, containing less than about 0.1 percent by high performance liquid chromatography of an impurity having the formula:

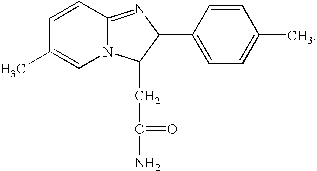

16. Zolpidem or a salt thereof of claim 12, containing less than about 0.1 percent by high performance liquid chromatography of an impurity having the formula:

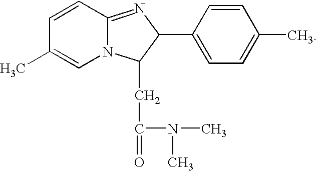

17. Zolpidem or a salt thereof of claim 12, containing less than about 0.1 percent by high performance liquid chromatography of an impurity having the formula: [chemical structure]
